(19) United States
(12) Patent Application Publication
(10) Pub. No.: US 2014/0255974 A1
(43) Pub. Date: Sep. 11, 2014

(54) Method and Use for the Detection of a Component of Interest in Biological Samples

(71) Applicant: Forschungsinstitut für Mineralische und Metallische Werkstoffe Edelsteine/Edelmetalle GmbH, Idar-Oberstein (DE)

(72) Inventors: Daniel Rytz, Herborn (DE); Sebastian Schwung, Munster (DE); Lothar Ackermann, Idar-Oberstein (DE)

(73) Assignee: Forschungsinstitut für Mineralische und Metallische Werkstoffe Edelsteine/Edelmetalle GmbH, Idar-Oberstein (DE)

(21) Appl. No.: 14/350,904

(22) PCT Filed: Sep. 20, 2012
(86) PCT No.: PCT/EP2012/068573

§ 371 Date: Apr. 10, 2014

(30) Foreign Application Priority Data
Oct. 11, 2011 (DE) 10 2011 054 382.1

(51) Int. Cl. G01N 33/50

(52) U.S. Cl. CPC G01N 33/5091
USPC 435/29

(57) ABSTRACT

The present invention relates to a use of bismuth ferrite crystals to detect a part of interest in biological samples. In order to provide a method which is cost-effective and in which a measurement can be carried out with a high degree of accuracy and which is quantifiable, the invention proposed that the part is marked with one or more bismuth ferrite crystals in a biological sample to be examined and the part marked in this manner is detected in the biological sample using at least one magnetic measuring method and at least on one optical measuring method.

The invention concerns the use of Bismuth ferrite crystals for the detection of a component of interest in biological samples.

For the detection of components in biological samples. classical imaging techniques such as nuclear magnetic resonance, ultrasound, positron emission tomography or optical coherence tomography are known and used in various fields. Restrictions of these techniques are caused by high costs or by technical limits, for example in the achievable resolution.

Optical imaging techniques are also used for the detection of components in biological samples, especially single cells, layers of single cells and tissue slices. They are able to overcome some of these restrictions. For example, techniques based on so-called “labels” (in the sense of optical markers) are in use, where single cells or groups of cells or parts within these groups can be coupled with such markers. Markers being used are constituted by semiconducting nanocrystals or semiconducting “quantum dots”, organic dyes, fluorescent pigments, etc.

In particular, imaging techniques where fluorescent dyes are used as markers are known. Fluorescent nanoparticles have several disadvantages: the fluorescence signal is proportional to the intensity of the excitation source only over a limited range. This saturation effect limits the maximum achievable intensity of the detected signal. Moreover, fluorescent materials exhibit aging effects which lead to bleaching and thus to a loss in sensitivity which depends on the time interval elapsed since the beginning of the measurement. In addition, the autofluorescence of other compounds which may be present in the samples to be investigated is an additional limitation of the methods based on fluorescence. Moreover, biological material is in some cases penetrated only poorly by electromagnetic waves in the spectral range between 350 and 750 nm, which is an important range for fluorescence measurements.

With this background in mind, the goal consists in providing a method for the detection of components in biological samples which is cost efficient, which allows measurements to be performed with high accuracy and which is quantitative.

This goal is reached with the use of Bismuth ferrite crystals for the detection of a component of interest in biological samples, provided that
the component in the biological sample to be analyzed is labelled with one or several Bismuth ferrite crystals and
the component labelled in this way is detected in the biological sample by at least one magnetic and one optical measurement technique.

Surprisingly, it has been shown that, when using Bismuth ferrite crystals, a detection of components in biological samples labelled with Bismuth ferrite is possible based on an optical measurement technique as well as on a magnetic detection scheme. By a combination of the optical measurement technique where preferably the nonlinear optical properties, especially the ability to generate the second harmonic (SHG), third harmonic or other wavelengths achievable through sum frequency generation, are used, and a magnetic measurement technique where the magnetic properties of Bismuth ferrites are involved, an improved sensitivity of the measurements can be achieved, resulting in a higher level of precision compared to a detection scheme of said components based on a single technique alone. Moreover, both detection techniques are implemented based on a single labelling with Bismuth ferrite crystals, thereby avoiding the need for a complicated double labelling approach of the components, where potentially steric issues could be expected, i.e., issues related to the detection or even to the labelling ability of the component in the biological sample.

In addition, magnetic forces can be used to position the crystals and nanocrystals.

In an embodiment of the invention, the optical measurement technique is an optical imaging process based on the nonlinear optical properties of the crystal, where a wavelength resulting from second harmonic, third harmonic or sum-frequency conversion of the incident wavelength is generated and detected.

In an embodiment of the invention, the magnetic measurement technique is magnetic resonance tomography.

When used as nonlinear optical probes, crystals, nanocrystals or nanoparticles, Bismuth ferrite crystals show decisive advantages as no intensity saturation, no aging and no self-fluorescence effects appear. Moreover, based on the nonlinear optical properties of Bismuth ferrite crystals, the second harmonic, third harmonic of sum frequency generated wave can be obtained from an incident electromagnetic wave and detected, the SHG, THG or SFG signal appearing in a wavelength range very distinct from the incident excitation or fundamental wave, and with the generation of this signal being achievable over an extended bandwidth of the fundamental wave.

It has been shown that Bismuth ferrite crystals are antiferromagnetic in the sense of this invention, with a Néel temperature T_{N}=370° C. This material shows a coexistence at room temperature of electric polarization and magnetic ordering. The antiferromagnetism of Bismuth ferrite can be described by a rotation of the Fe spins similar to a cycloid. The spatial periodicity of the cycloid amounts to 62 nm. This leads to superparamagnetic properties of Bismuth ferrite crystals which can be used for magnetic imaging techniques.

For the application described here, the magnetic properties of Bismuth ferrite constitute an essential advantage when simultaneously magnetic and optical imaging techniques of molecules, cells, tissues or entire organisms are targeted. Simultaneous imaging techniques have the practical advantage that correlations between measurements based on different physical phenomena strongly improve the resolution and the identification ability. In the case of the materials used so far for the detection of components of interest in biological samples, useful magnetic properties or, respectively, additional properties allowing detection measurements with distinct techniques based on a single label are lacking.

In an embodiment of the invention, the implementation of Bismuth ferrite crystals in magnetic imaging techniques follows from their use as contrast improving agents for MRI (“magnetic resonance imaging”). The magnetic Bismuth ferrite particles exhibit a relatively high saturation magnetization in the range between 1 and 6 emu/g. In absence of a magnetic field, however, no permanent magnetic moment is present. Superparamagnetic Bismuth ferrite can lead to a locally increased relaxation rate, thereby generating additional contrast in the MRI image: at the site where Bismuth ferrite is located, an especially dark or bright image contrast appears.

In an embodiment of the invention, based on the use of Bismuth ferrite crystals, MRI and SHG images can be obtained simultaneously in a so-called multimodal technique. In this way a method is implemented where several imaging techniques can be used at the same time on one specific sample with only one label. The interpretation of the images can thus be performed with a high confidence level, due to the fact that different modes for image generation can be used for cross checking and controlling.

In an embodiment of the invention, a spatial localization of the crystals is obtained, for example, from the simultaneous detection based on SHG enhanced microscopy and magnetic resonance. Because the Bismuth ferrite crystals were selectively attached to cells or organisms, the analysis of the detected crystals allows a three dimensional visualization of the targeted cells or organisms and, on the other hand, a direct correlation between two fundamentally different measurements.

Bismuth ferrite crystals in the sense defined in the present invention encompass pure BiFeO₃-crystals as well as mixed crystals with BiFeO₃ as an end member. The term mixed crystals with BiFeO₃ as an end member or Bismuth ferrite crystals, respectively, does not exclude the fact that, in the mixed crystal, the proportion of one or several additional crystals is not included or is only included at a very low level.

In specific embodiments of the invention, mixed crystals with BiFeO₃ as an end member or pure BiFeO₃ crystals are used with a BiFeO₃ concentration of at least 40 mol %, preferably at least 50 mol %, more preferably at least 70 mol %, and most preferably at least 80 mol % and up to 100 mol %. A measurement of the purity can be performed by X-rays crystallography in one embodiment. Pure BiFeO₃ crystals, as the term is defined here, describe crystals which are essentially composed of BiFeO₃ and include only a small amount of unavoidable impurities.

In one embodiment of the invention, the properties of BiFeO₃ (as indicated here for bulk samples or single crystals) are:
Crystal symmetry: R3c (rhombohedral)
Lattice parameters: a=b=5.571 A, c=13.858 A.
Ferroelectric polarization: P_{S}=60 μC/cm²
Coercive field: E_{c}=12 kV/cm

Preferably the used crystals show one, several or all of the properties mentioned above.

The properties of Bismuth ferrite in the form of nanocrystals can deviate with respect to these values. In practice, however, it has been demonstrated that Bismuth ferrite crystals are ferroelectric and thus non-centrosymmetric.

In this context, the term “biological samples” designates isolated cells, cell layers, samples extracted from cells, organs, tissue as well as small organisms and parts of organisms, including certain parts of the skin as well as parts near lesions and additional parts which are accessible to direct radiation, for example from a laser, naturally or following a (surgical) operation.

In one embodiment of the present invention, the biological sample is selected among single cells, cell membranes, nucleotides, neuronal cells, tissue slices, organ biopsies or entire organisms as well as slices from organisms, such as lesions or surgery areas, in special cases also single molecules.

In one embodiment of the present invention, the component of interest in the biological sample is a cell, especially cancer cells or stem cells, a cell organ, a molecule, a cluster of molecules or an assembly of molecules.

In one embodiment of the present invention, the component of interest is a molecule, selected from proteins, parts of proteins, DNA and RNA or a part of a molecule such as nucleotides, amino-acids or peptides.

In one embodiment of the present invention, the Bismuth ferrite crystals are brought in the immediate vicinity of the biological sample. In certain embodiments, the crystals can be brought into the cells or other biological samples in an aqueous suspension.

In one embodiment of the present invention, before being used as labels for components in biological samples, the Bismuth ferrite crystals are embedded in a polymer, characterized in that the polymer is preferably selected from a group including dextran, dextran modified with carboxyl or amino groups, polyethyleneglycol (PEG) or amino-PEG.

In the case of such embedded Bismuth ferrite crystals, the surface is modified by a coating, for example a dextran, modified by carboxyl or amino groups, coating. Polyethyleneglycol (PEG) or amino-PEG coatings appear to be especially favorable. In this case, the coating can on one side strongly limit the agglomeration of Bismuth crystals, and on the other a functionalization of the Bismuth ferrite crystals can be implemented, as described below. For the functionalization, the embedded crystals are connected with a specific substance that allows to link the Bismuth ferrite crystals to a component of interest such as a defined targeted substance or group of cells: the coating is in this case used to functionalize the crystals, which thereby can be analytically used as labelling material for subsequent detection of specific components of interest.

In an embodiment of this invention, the polymer is additionally linked with a binding molecule which is able to bind itself to one or more components of interest in the biological sample and which is characterized in that it is selected from a group comprising antibodies, substrates and receptor agonists, as well as analogs to the previously mentioned small peptides, tumor specific proteins, dextrans, modified dextrans, glycosyl chains, amino and carboxyl groups. Techniques for embedding and binding of crystals and nanocrystals in general are known to persons skilled in the art and can be used in the case of the application of Bismuth ferrite crystals claimed here.

In an embodiment of the present invention, the Bismuth ferrite crystal is linked with a binding molecule characterized in that it is selected from antibodies, substrates and receptor agonists as well as analogs to the previously mentioned small peptides, tumor specific proteins, dextrans, modified dextrans, glycolsyl chains, amino and carboxyl groups. A direct link also encompasses the binding by network building molecules, including carbodiimides, esters, imide esters, etc., in forms known to the person skilled in the art.

In an embodiment of the present invention, the Bismuth ferrite crystals have a phase purity of more than 90 mol %, preferably more than 93 mol %, as measured by X-rays crystallography. Based on crystals with such purity, especially good signals can be obtained.

In an embodiment of the present invention, the Bismuth ferrite crystals have an average particle size from 5 to 1000 nm, preferably from 25 to 350 nm, and more preferably from 30 to 125 nm. Crystals with such a particle size can easily be directly or indirectly injected into biological samples, for example after embedding in additional materials. Moreover, they exhibit a size which renders them easily detectable by optical as well as magnetic imaging techniques. It should be noted that superparamagnetic properties as described above have been demonstrated with these typical sizes.

In one embodiment of the present invention, the Bismuth ferrite crystals follow the general formula I

(BiFeO₃)_{1-x-y}(ABO₃)ₓ(A′B′O₃)_{y} (Formula I)

or the different but equivalent formula II

Bi_{1-x-y}AₓA′_{y}Fe_{1-x-y}BₓB′_{y}O₃ (Formula II)

wherein:

A and A′ are selected independently from one another from the group consisting of Pb, Fe, La, Y, Gd, Bi, Ba, K, Na, K_{0.5}Bi_{0.5} and Na_{0.5}Bi_{0.5}, where, if A and A′ designate K_{0.5}Bi_{0.5} or Na_{0.5}Bi_{0.5}, then the other member of A and A′ is not chosen among K_{0.5}Bi_{0.5} and Na_{0.5}Bi_{0.5},

B and B′ are selected independently from one another from the group consisting of Ti, Sc, Al, Ga, Fe, Mn, Cr, Co, Nb,

x and y have independently from one another a value from 0 to 0.5 and the sum x+y equals a value from 0 to 0.5.

In an embodiment of the present invention, the Bismuth ferrite crystals are selected among:
BiFeO₃—PbTiO₃=(1−x)BiFeO₃+xPbTiO₃,
BiFeO₃—BiScO₃=(1−x)BiFeO₃+xBiScO₃,
BiFeO₃—FeAlO₃=(1−x)BiFeO₃+xFeAlO₃,
BiFeO₃—FeGaO₃=(1−x)BiFeO₃+xFeGaO₃,
BiFeO₃—FeScO₃=(1−x)BiFeO₃+xFeScO₃,
BiFeO₃—LaFeO₃=(1−x)BiFeO₃+xLaFeO₃,
BiFeO₃—YFeO₃=(1−x)BiFeO₃+xYFeO₃,
BiFeO₃—GdFeO₃=(1−x)BiFeO₃+xGdFeO₃,
BiFeO₃—BiMnO₃=(1−x)BiFeO₃+xBiMnO₃,
BiFeO₃—BiCrO₃=(1−x)BiFeO₃+xBiCrO₃,
BiFeO₃—BaTiO₃=(1−x)BiFeO₃+xBaTiO₃,
BiFeO₃—KNbO₃=(1−x)BiFeO₃+xKNbO₃,
BiFeO₃—NBT=(1−x)BiFeO₃+xNa_{1/2}Bi_{1/2}TiO₃,
BiFeO₃—KBT=(1−x)BiFeO₃+xK_{1/2}Bi_{1/2}TiO₃
BiFeO₃—NBT-KBT=(1−x−y)BiFeO₃+xNa_{1/2}Bi_{1/2}TiO₃+yK_{1/2}Bi_{1/2}TiO₃
   where x is a number from 0 to 0.5, preferably from 0 to 0.4. The bracketing values are preferably included.

This list can obviously be extended and many additional combinations with Bismuth ferrite (for example, ternaries such as BiFeO₃—NBT-KBT) are possible and are also part of this invention.

In one embodiment of the present invention, the Bismuth ferrite crystals are treated with a Fe₃O₄ containing coating.

The term Fe₃O₄-containing coating in the sense of this invention encompasses coatings which preferably contain a Fe₃O₄ fraction of at least 80 mol % and more preferably a Fe₃O₄ fraction of up to 100 mol %.

A coating of this type enhances the magnetization of the Bismuth ferrite crystals, thereby markedly improving the detection capability of a magnetic measurement technique and enhancing the contrast. An enhanced magnetization implies a stronger influence on the relaxation times and thus an improvement of the contrast from MRT measurements. The contrast of Bismuth ferrite crystals coated along those lines obtained in MRT measurements is comparable to the contrast that one can obtain with iron oxide. The magnetization acts directly onto the protons in the vicinity of the coating. An additional advantage of such coated Bismuth ferrite crystals is that the efficiency of the second harmonic generation in optical imaging techniques is maintained in the sense that the coating does not negatively impact this measurement. Moreover, iron oxide is not toxic and therefore usable without (intense) side effects in living systems, such as, for example, cells or other biological samples.

It is understood that in specific embodiments of the present invention, other embedding or coating techniques such as generally available for Bismuth ferrite crystals can exist independently from a Fe₃O₄ containing coating.

In an embodiment of the present invention, the thickness of the Fe₃O₄ containing coating is in the range from 30 to 50 nm

In an embodiment of the present invention, the at least one optical measurement technique is a technique where a laser beam with a first wavelength is illuminating a biological sample and a signal with a second wavelength is measured, which is reflected by the biological sample, the second wavelength being ½ of the first wavelength and the first wavelength being in a wavelength range of preferably from 1800 to 500 nm, particularly preferably from 1640 to 1560 nm or from 1070 to 1010 nm.

Using Bismuth ferrite crystals generating a second harmonic as a labelling agent for an optical imaging technique provides an alternative to fluorescence microscopy. When illuminated by an intense laser beam as the fundamental wave with a specific wavelength, in every crystal light is generated with the corresponding halved wavelength. When Bismuth ferrite crystals are linked as markers to components of interest in a biological sample, detecting such molecules in cells, tissues or entire organisms becomes possible.

In an embodiment of the invention, the at least one optical measurement technique is a technique where two laser beams with a first and second wavelength, respectively, are illuminating a biological sample and a signal with a third wavelength is measured, which is reflected by the biological sample, the third wavelength corresponding to the sum frequency of the first and second wavelengths, and the first two wavelengths range ranging in a preferred wavelength interval from 1800 to 500 nm.

In an embodiment of the invention, the at least one optical measurement technique is a technique where a laser beam with a first wavelength is illuminating a biological sample and a signal with a second wavelength is measured, which is reflected by the biological sample, the second wave-length being ⅓ of the first wavelength and the first wavelength ranging in a preferred wavelength interval from 1800 to 500 nm.

Based on Bismuth ferrite crystals, SHG (“second harmonic generation”) signals can be demonstrated: under illumination by a laser source with a peak energy at a wavelength lambda. Single Bismuth ferrite crystals can be detected, because every crystal doubles the frequency of a fraction of the incident energy, i.e., every crystal generates light at a wavelength lambda/2.

In certain embodiments of the invention, the following wavelength combinations can be used:

| Fundamental wave | SHG-wave |
|---|---|
| (wavelength = lambda, in nm) | (wavelength = lambda/2, in nm) |
| 1800-1400 | 900-700 |
| 1640-1560 | 820-780 |
| 1600 | 800 |
| 1580 | 790 |
| 1400-1200 | 700-600 |
| 1350-1290 | 675-645 |
| 1310 | 655 |
| 1200-800 | 600-400 |
| 1070-1010 | 535-505 |
| 1064 | 532 |
| 1030 | 515 |
| 800-500 | 400-250 |

Based on similar principles, in certain embodiments of the invention, one can as described above combine two wavelengths lambda 1 and lambda 2, and due to analogous nonlinear optical effects, a third wavelength lambda 3 is obtained by sum frequency conversion, according to: 1/lambda 3=1/lambda 1+1/lambda 2.

In a special case of sum frequency conversion, the fundamental wave lambda 1=lambda is combined with its second harmonic lambda 2=lambda/2 and the generated wavelength is the third harmonic lambda 3=lambda/3. This THG imaging technique (“third harmonic generation”) is comparable to the SHG technique. Several THG wavelengths (with rounded values) are listed in the following table.

| Fundamental wave | THG-wave |
|---|---|
| (wavelength = lambda, in nm) | (wavelength = lambda/3, in nm) |
| 1800-1200 | 600-400 |
| 1650-1500 | 550-500 |
| 1350-1290 | 450-430 |
| 1320 | 440 |
| 1110-990 | 370-330 |
| 1064 | 355 |
| 1030 | 343 |
| 960-750 | 320-250 |

In one embodiment of the present invention, the magnetic measurement technique is a process where the biological sample is measured and preferably imaged based on the observed relaxation signals by MRI (“magnetic resonance imaging”) in a magnetic field with a magnetic flux density from 0.001 to 60 Tesla, and preferably from 0.01 to 4 Tesla.

For the described application, the magnetic properties of the Bismuth ferrite crystals present essential advantages in order to achieve the goal of implementing simultaneous magnetic and optical imaging techniques for molecules, cells or entire organisms. In practice, the advantage of simultaneous imaging techniques is the result of correlations based on different physical effects which strongly improve the resolution and the identification capabilities. Among the materials used so far for the detection of components of interest in biological samples, usable magnetic properties or an additional property leading to detection possibilities with a single labelling agent are lacking.

Bismuth ferrite crystals can be used in certain embodiments for SHG and magnetic measurements, tests and imaging techniques. Preferably, the SHG tests are performed with a laser beam (fundamental wave) in the range 1800-500 nm, with specially emphasized ranges 1800-1400 nm and 1100-700 nm for technical reasons: this corresponds to detected SHG wavelengths in the ranges 900-250 nm, 900-700 nm and 550-350 nm. The Bismuth ferrite particles exhibit superparamagnetic behavior with a saturated magnetization between 0.3 and 15 emu/g.

Optical imaging of the Bismuth ferrite crystals is performed in a multiphoton microscope coupled to a short pulse laser source. This source can be, for example, a Ti:sapphire oscillator which emits pulses in the wavelength range 700-1100 nm with a 60-120 fs pulse duration and a 60-100 MHz repetition rate. One advantage of a Ti:sapphire oscillator is its tuning capability, thus making measurements at different wavelengths possible. Other short pulse lasers, however, can also be used, such as, for example, fiber lasers based on Yb³⁺ or Er³⁺. It has been shown that the efficiency of the nonlinear optical processes in Bismuth ferrite crystals is even higher than in crystals such as BaTiO₃, LiNbO₃, KNbO₃, KTP or ZnO which are conventionally used for this purpose.

The magnetic properties with imaging capability of Bismuth ferrite crystals are responsible for an image contrast based on the relaxation times in magnetic resonance tomography (MRT). The small sized particles of Bismuth ferrite crystals (smaller than 500 nm, preferably smaller than 250 nm) exhibit a superparamagnetic magnetization behavior with a saturable magnetization between 0.3 and 15 emu/g. Even if this value is significantly lower than the magnetization of commercially available iron oxide particles used in the MRT or MRI (“magnetic resonance imaging”) imaging technique, measurements can also be performed with Bismuth ferrite particles. Values between 900 and 1500 ms were determined for the relaxation time T1 at 37° C. at 30-60 MHz. The values for T2 were between 200 and 600 ms. Thus such measurements can also be performed with Bismuth ferrite particles.

The object of the invention mentioned in the introduction is also achieved by a method for imaging biological cells and tissues by optical and magnetic techniques using Bismuth ferrite crystals as well as related materials. According to the invention, material compositions are described which allow simultaneous optical and magnetic detection processes in biological samples. The synthesis of useful Bismuth ferrite crystals, nanocrystals or nanoparticles is described here.

The object of the invention is also achieved by providing crystals for multimodal imaging techniques based on the generation of the second harmonic of laser light (the so-called SHG technique): these crystals, nanocrystals or nanoparticles contain Bismuth ferrite (BiFeO₃ or BFO) with ferroelectric and magnetic properties suitable for various optical and magnetic techniques.

### EXAMPLES

### 1. Preparation of Bismuth Ferrite Crystals and Measurement

The different steps from the synthesis to the application of Bismuth ferrite nanocrystals having the desired properties can be summarized as an example in the following:
(1) synthesis of Bismuth ferrite with particle sizes in the range from 5 to 1000 nm;
(2) optional embedding of the Bismuth ferrite crystals in functionalized polymers for optimal dispersion in a solution, functionalization of the polymer coating of the embedded Bismuth ferrite crystal as a preparation step for selective linking to a component of interest in a biological sample, for example to a molecule, cell or tissue to be detected, and/or functionalization of the polymer coating as a preparation step for additional measurement and sensing properties such as, for example, positron emitters that can be additionally attached.
(3) bringing the biological sample in contact with a solution containing the Bismuth ferrite crystals described above, for example an aqueous dispersion of Bismuth ferrite crystals;
(4) illumination of the sample with laser radiation in the wavelength range 500-1800 nm and detection of the frequency converted, preferably of the second harmonic signals;
(5) detection of the magnetic properties;
(6) processing of the resulting data by simultaneously converting the nonlinear optical and the magnetic signals to images, in order to establish correlations between the two techniques; thereby an imaging technique based on several modes is achieved.

### 2. Optical Measurement Technique

Using Bismuth ferrite crystals, SHG (i.e., “second harmonic generation”) signals can be detected: the trigger being a laser source with high peak power at a wavelength lambda. Single Bismuth ferrite crystals with the indicated sizes can be detected, as every crystal converts the frequency of a fraction of the incident radiation, i.e., every crystal generates light at a wavelength lambda/2. As examples, the following wavelength combinations can be used:

| Fundamental wave | SHG-wave |
|---|---|
| (wavelength = lambda, in nm) | (wavelength = lambda/2, in nm) |
| 1800-1400 | 900-700 |
| 1640-1560 | 820-780 |
| 1600 | 800 |
| 1580 | 790 |
| 1400-1200 | 700-600 |
| 1350-1290 | 675-645 |
| 1310 | 655 |
| 1200-800 | 600-400 |
| 1070-1010 | 535-505 |
| 1064 | 532 |
| 1030 | 515 |
| 800-500 | 400-250 |

Following a similar principle, two wavelengths lambda 1 and lambda 2 can be combined and a third wavelength lambda 3 generated by similar nonlinear optical effects via sum frequency generation:

the following being valid 1/lambda 3=1/lambda 1+1/lambda 2.

In a special case of sum frequency generation, the fundamental wave lambda 1=lambda and its second harmonic lambda 2=lambda/2 are combined to generate a wavelength which is the third harmonic lambda 3=lambda/3. This THG imaging technique is essentially comparable to the SHG technique. Several possible THG wavelengths (with rounded values) are summarized in the following table.

| Fundamental wave | THG wave |
|---|---|
| (wavelength = lambda, in nm) | (wavelength = lambda/3, in nm) |
| 1800-1200 | 600-400 |
| 1650-1500 | 550-500 |
| 1350-1290 | 450-430 |
| 1320 | 440 |
| 1110-990 | 370-330 |
| 1064 | 355 |
| 1030 | 343 |
| 960-750 | 320-250 |

Using Bismuth ferrite crystals generating a second harmonic as a labelling agent for an optical imaging technique provides an alternative to fluorescence microscopy. When illuminated by an intense laser beam as the fundamental wave with a specific wavelength, in every crystal light is generated with the corresponding halved wavelength. When Bismuth ferrite crystals are linked as markers to components of interest in a biological samples, detecting such molecules in cells, tissues or entire organisms becomes possible

Optical imaging of the Bismuth ferrite crystals is performed for example in a multiphoton microscope coupled to a short pulse laser source. This source can be, for example, a Ti:sapphire oscillator which emits pulses in the wavelength range 700-1100 nm with a 60-120 fs pulse duration and a 60-100 MHz repetition rate. One advantage of a Ti:sapphire oscillator is its tuning capability, thus making measurements at different wavelengths possible. Other short pulse lasers, however, can also be used, such as, for example, fiber lasers based on Yb³⁺ or Er³⁺. It has been shown that the efficiency of the nonlinear optical processes in Bismuth ferrite crystals is even higher than in crystals such as BaTiO₃, LiNbO₃, KNbO₃, KTP or ZnO which are conventionally used for this purpose. Available images obtained in a multiphoton microscope using Bismuth ferrite crystals show the presence of single Bismuth ferrite crystals on cell membranes and inside the cytoplasm.

### 3. Magnetic Measurement Technique

The magnetic properties with imaging capability of Bismuth ferrite crystals are responsible for an image contrast based on the relaxation times in magnetic resonance tomography (MRT). The small sized particles of Bismuth ferrite crystals (smaller than 500 nm, preferably smaller than 250 nm) exhibit a superparamagnetic magnetization behavior with a saturable magnetization between 0.3 and 15 emu/g. Even if this value is significantly lower than the magnetization of commercially available iron oxide particles used in the MRT or MRI (“magnetic resonance imaging”) imaging technique, measurements can also be performed with Bismuth ferrite particles. Values between 900 and 1500 ms were determined for the relaxation time T1 at 37° C. at 30-60 MHz. The values for T2 were between 200 and 600 ms. Thus such measurements can also be performed with Bismuth ferrite particles.

In an additional field of applications, magnetic forces can be used to position Bismuth ferrite crystals.

### 4. Bismuth Ferrite Crystals

Bismuth ferrite (BiFeO₃ or BFO) is one of three stable phases in the Fe₂O₃—Bi₂O₃ system. Taking x as the molar concentration of Bi₂O₃ defined as x=[Bi₂O₃] ([Bi₂O₃] [Fe₂O₃]), then Bismuth ferrite corresponds to a phase with x=0.50. One finds the neighboring phases Bi₂Fe₄O₉ (with x=0.333) and Bi₂₅FeO₃₉ (x=0.94). The Bismuth ferrite phase is non congruently melting and is stabilized by excess Bi₂O₃ in a high temperature solution: the stability range lies between 925° C. (for x=0.60) and 777° C. (for x=0.86).

In the invention, Bismuth ferrite crystals are used where one has determined that below approx. 825° C. Bismuth ferrite has a non centrosymmetric crystal symmetry. Between 825 and 925° C., Bismuth ferrite is centrosymmetric, above 925° C., another phase possibly appears. Depending on the reference in the literature, the various phase transition temperatures vary widely. The neighboring phases Bi₂Fe₄O₉ and Bi₂₅FeO₃₉ are both centrosymmetric and thus unfavorable as SHG materials.

In one embodiment of the invention, the properties of Bismuth ferrite near 22° C. (indicated here for bulk samples or single crystals) are:
Crystal symmetry: R3c (rhombohedral)
Lattice parameters: a=b=5.571 A, c=13.858 A.
Ferroelelectric Polarization: P_{S}=60 μC/cm²
Coercive field: E_{c}=12 kV/cm

Preferably, the used crystals exhibit one, several or all of the properties mentioned above.

The properties of Bismuth ferrite in the form of nanocrystals can deviate from these values. As a general principle, however, it has been shown that Bismuth ferrite nanocrystals are ferroelectric and thus non centrosymmetric.

Using Bismuth ferrite crystals, measurements with laser sources at 1064 nm and 800 nm have confirmed the favorable efficiency of Bismuth ferrite for the generation of corresponding SHG signals. In addition, it could be demonstrated that Bismuth ferrite crystals are especially suitable for an optical imaging technique based on second harmonic generation. In an example, a short pulse laser with energy pulses shorter than 10⁻⁸ seconds is focused onto a sample containing Bismuth ferrite crystals. The focus of the laser beam scans the sample following a defined pattern. The harmonic wave generated by the Bismuth ferrite crystals is collected by the focusing objective or by an additional second objective arranged before the focusing objective, and is imaged on several detectors, such as for example photomultipliers or avalanche photodiodes. Before reaching the detectors, the incident light is filtered spectrally. Three dimensional images are obtained when the focus locations and the SHG intensity are correlated for every measurement point.

The SHG imaging technique can be performed with various types of Bismuth ferrite crystals which are described as examples in the following. The usable laser wavelengths can be freely chosen between lambda=1800 and 500 nm, as already mentioned. The imaging technique can also be implemented with other (than the second harmonic at lambda/2) wavelengths to be detected: for example, the third harmonic at lambda/3 or the wavelength generated by the sum frequency of several lasers can also be analyzed and used for the generation of spatial images.

For magnetic and optical imaging techniques of molecules, cells, tissues or entire organisms, nanocrystals of Bismuth ferrite but also mixed BiFeO₃-A′B′O₃*A″B″O₃ materials are usable. Several examples in the following list explain which A′, B′, A″, B″ ions can be used:
BiFeO₃—PbTiO₃=(1−x)BiFeO₃+xPbTiO₃
BiFeO₃−BiScO₃=(1−x)BiFeO₃+xBiScO₃
BiFeO₃—FeAlO₃=(1−x)BiFeO₃+xFeAlO₃
BiFeO₃—FeGaO₃=(1−x)BiFeO₃+xFeGaO₃
BiFeO₃—FeScO₃=(1−x)BiFeO₃+xFeScO₃
BiFeO₃—LaFeO₃=(1−x)BiFeO₃+xLaFeO₃
BiFeO₃—YFeO₃=(1−x)BiFeO₃+xYFeO₃
BiFeO₃—GdFeO₃=(1−x)BiFeO₃+xGdFeO₃
BiFeO₃—BiMnO₃=(1−x)BiFeO₃+xBiMnO₃
BiFeO₃—BiCrO₃=(1−x)BiFeO₃+xBiCrO₃
BiFeO₃—BaTiO₃=(1−x)BiFeO₃+xBaTiO₃
BiFeO₃—KNbO₃=(1−x)BiFeO₃+xKNbO₃
BiFeO₃—NBT=(1−x)BiFeO₃+xNa_{1/2}Bi_{1/2}TiO₃
BiFeO₃—KBT=(1−x)BiFeO₃+xK_{1/2}Bi_{1/2}TiO₃
BiFeO₃—NBT-KBT=(1−x−y)BiFeO₃+xNa_{1/2}Bi_{1/2}TiO₃+y K_{1/2}Bi_{1/2}TiO₃

This list can obviously be expanded and many additional combinations with Bismuth ferrite (for example, ternaries such as BiFeO₃—NBT-KBT) are possible and also included in this invention.

Bismuth ferrite crystals can be used for SHG and magnetic measurements, tests and imaging processes. Preferably, SHG tests are performed with (fundamental wavelength) laser beams in the range 1800-500 nm, with the ranges 1800-1400 nm, 1100-700 nm being of special technical importance: these correspond to detected SHG wavelengths in the ranges 900-250 nm, 900-700 nm and 550-350 nm. The Bismuth ferrite particles exhibit superparamagnetic behavior with a saturable magnetization between 0.3 and 15 emu/g.

### 5. Synthesis of Bismuth Ferrite Crystals

The synthesis of Bismuth ferrite including Bismuth ferrite mixed crystals can be implemented with several methods. In the following descriptions, the synthesis processes for Bismuth ferrite and Bismuth ferrite crystals, respectively, can be adapted without difficulties by a person skilled in the art to the synthesis of Bismuth ferrite mixed crystals.

### Milling of Bismuth Ferrite Single Crystals or Ceramics:

In this method, fragments of single or poly-crystals are processed by an automatic mill or a hand operated mortar with corresponding pestle. An example for the operating parameters for a mechanical mill is as follows:
milling in containers made of silicon nitride.
preprocessing with milling spheres made of silicon nitride in water during 3 min at 850 rpm.
freeze drying of preprocessed powder in order to reduce water content.
main processing with milling spheres made of zirconium oxide in water during 5 min at 1100 rpm.
freeze drying of powder.

Following these steps, the resulting Bismuth ferrite powder is either directly used for measurements or it is refined by decantation. Bismuth ferrite with particle sizes under 200 nm have been obtained and used for optical and magnetic measurements. For particles in the range between 50 and 150 nm, a SHG signal was observed from single particles. The Bismuth ferrite crystalline particles exhibit a superparamagnetic behavior with a saturable magnetization between 0.3 and 15 emu/g.

Pechini Method with Bi₂O₃ and Fe(CH₃OO)₂ as Starting Materials:

In this method, ionic starting materials are initially dissolved. Subsequently, chelating agents (a citrate, for example) able to polycondensate are added. Polycondensation occurs due to heating. At a later stage, the polycondensates are decomposed at high temperatures.

The method described here comprises the following steps:

Step 1: while continuously stirring and heating to the boiling point, Bi₂O₃ is dissolved in hot nitric acid (20% HNO₃).

Step 2: Fe(CH₃O₀)₂ is added to the hot, transparent solution.

Step 3: addition of chelating agent.

Step 4: a gel is formed by evaporation of the solvents in a magnetic stirrer.

Step 5: heating of the powder to 400° C. in air in an Al₂O₃ crucible during 3 Std.

Step 6: mill precursors obtained in step 5 in mortar and heat to various temperature levels (between 500 and 800° C.) and keep at temperature during 1 to 8 hours.

High purity Bismuth ferrite crystals are obtained.

Pechini method with Bi(NO₃)₃*5H₂O and Fe(NO₃)₃*9H₂O as starting materials:

In this further method, the starting materials Bi(NO₃)₃*5H₂O and Fe(NO₃)₃*9H₂O are initially dissolved in dilute nitric acid (10% HNO₃), in order to prevent the formation of Bismuth oxynitrate. Then the addition of a polycondensate enabling chelating agent follows. The polycondensation occurs by heating. In a later stage, the polycondensates are decomposed at high temperature. The method described here comprises the following steps:

Step 1: while continuously stirring and heating to the boiling point, Bi(NO₃)₃*5H₂O and Fe(NO₃)₃*9H₂O are dissolved in hot nitric acid (10% HNO₃).

Step 2: addition of chelating agent. Various chelating agents can be used: citric acid, ethylendiaminetetraacetic acid (EDTA, Titriplex II), tris(hydroxymethyl)aminomethane. Combinations with PEG 300 or PEG 3000 are also possible, for example in the cases of citric acid or tris(hydroxymethyl)aminomethane.

Step 3: a gel is formed by evaporation of the solvents in a magnetic stirrer.

Step 4: heating of the powder to 400° C. in air in an Al₂O₃ crucible during 3 Std.

Step 5: mill precursors obtained in step 4 in mortar and heat to various temperature levels (between 500 and 800° C.) and keep at temperature during 1 to 8 hours. High purity Bismuth ferrite crystals are obtained.

The synthesis methods for Bismuth ferrite nanocrystals can readily be modified by a person skilled in the art. In the Pechini method, it is conceivable to change the starting materials, the required temperatures, the chelating agent and the embedding polymer (PEG, for example) and to obtain Bismuth ferrite crystals following many different experimental paths which are suited for imaging applications.

Additional fabrication methods such as hydrothermal synthesis, microwave combustion, among others, are also suitable.

The fabricated samples could all be successfully used for SHG and magnetic measurements, tests and imaging techniques. SHG tests were performed with a (fundamental) laser beam in the range 1800-500 nm; in addition, the ranges 1800-1400 nm, 1100-700 nm are technically especially significant: these correspond to detected SHG wavelengths in the ranges, 900-250 nm, 900-700 nm and 550-350 nm. As already mentioned in an example describing their synthesis, Bismuth ferrite particles exhibit superparamagnetic magnetization behavior with a saturated magnetization between 0.3 and 15 emu/g.

Based on simultaneous detection, for example by SHG enhanced microscopy and magnetic resonance, a spatial localization of the crystals is obtained. Because the crystals were selectively docked onto cells or organisms, the analysis of the detected crystals allows three dimensional imaging of the targeted cells or organisms on one hand and a direct correlation between two measurements based on different principles on the other. This correlation is only possible thanks to the unique properties of the materials synthesized in the invention and their first time use in the proposed measurement techniques.

### 6. Purity of Bismuth Ferrite Crystals

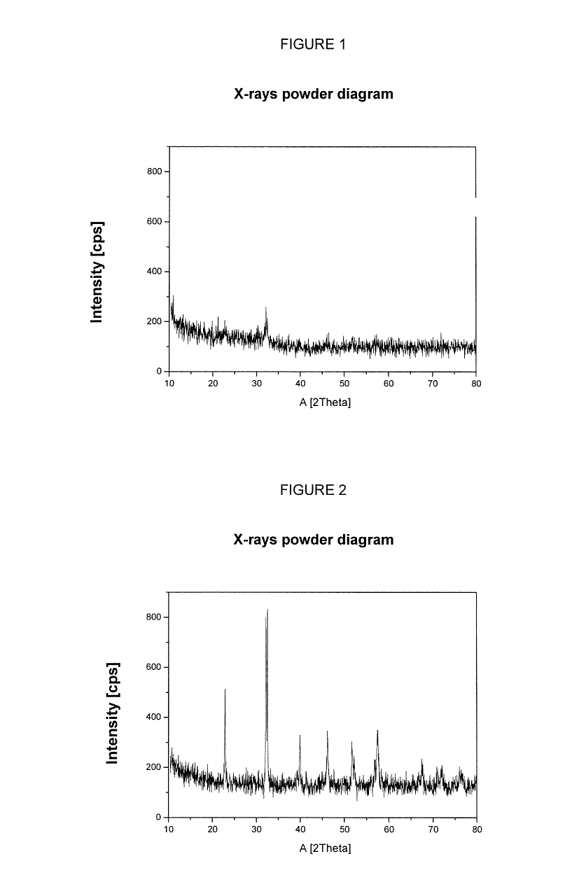

An example of a Bismuth ferrite sample with adequate phase purity can be seen in the X-rays powder diagrams in the FIGS. 1 and 2.

The measurements were performed with a SEIFERT C3000 instrument, equipped with a copper anode, without monochromator and without K_{β} filter. In the FIGS. 1 and 2, typical Bismuth ferrite powder diagrams are displayed, with the principal Bragg diffraction peaks at 2Theta angles characteristic for Bismuth ferrite at 22.82°, 32.44°, 32.54°, 40.04°, 40.18°, 46.6°, 52.44°, 52.56°, 57.88°, 57.94°, 58.1°, 67.94°, 68.14°, 72.7°, 72.8°, 73.0°, 77.36°, 77.52°. FIG. 1 shows the powder diagram of a sample treated at 400° C. in open air during 4 hours, whereas FIG. 2 shows the diagram of a sample treated at 600° C. The enhanced crystallinity achieved after treatment at 600° C. leads to the expected diagram with a markedly larger number of identifiable peaks. The measured powder diagrams were compared to and interpreted with the usual standard spectra (for example PCD 1910862 in the case of BFO).

### 7. Enhancing the Magnetization of Bismuth Ferrite Crystals

In order to improve the detection capabilities by a magnetic measurement technique, the magnetization of Bismuth ferrite crystals can be enhanced by several different methods:

Synthesis of Bismuth Ferrite Crystals with a Fe₃O₄ Coating Obtained by Coprecipitation of Fe₃O₄ on Bismuth Ferrite
a. Bismuth ferrite crystals obtained by one of the methods described above are dispersed in destillated water.
b. A soluble salt containing Fe²⁺ and/or Fe³⁺ such as iron(II/III)chloride, iron(II/III)nitrate, iron(II/III)acetate, is added.
c. By addition of NH₄OH the pH value is increased to 7.

Fe₃O₄ coated Bismuth ferrite crystals are obtained.

Synthesis of Bismuth Ferrite Crystals in a “Bottom-Up” Process with Magnetic Functionalization of the Surface by a Fe₃O₄ Coating.
a. Decomposition of suitable iron and Bismuth containing (iron(III)nitrate, iron acetate or Bismuth(III)nitrate, Bismuth oleate, respectively) in a solvent with a high boiling point such as oleic acid.
b. Addition of a Fe²⁺ and/or Fe³⁺ containing salt soluble in organic solvents such as iron(II/III)acetate or iron(II/III)oleate.
c. Cleaning of the nanoparticles by centrifugation and washing in acetone or ethanol.

The Bismuth ferrite crystals coated in such processes exhibit markedly higher magnetizations. At applied fields >30 kOe, the saturable magnetization reaches 25 emu/g, preferably 50 emu/g and more.

1. Method for the detection of a component of interest in biological samples, wherein
the component in the biological sample to be analyzed is labelled by one or more Bismuth ferrite crystals and
the labelled component is detected by at least one magnetic and at least one optical measurement technique.

2. Method as set forth in claim 1, wherein the Bismuth ferrite crystals have a phase purity of more than 90 mol %, preferably 93 mol %, as measured by X-rays crystallography.

3. Method as set forth in claim 1, wherein the Bismuth ferrite crystals show an average particle size from 5 to 1000 nm, preferably from 25 to 350 nm, more preferably from 30 to 125 nm.

4. Method as set forth in claim 1, characterized in that the Bismuth ferrite crystals have the following general formula 1:
(BiFeO₃)_{1-x-y}(ABO₃)ₓ(A′B′O₃)_{y} (Formula I)
or the general formula II
Bi_{1-x-y}AₓA′_{y}Fe_{1-x-y}BₓB′_{y}O₃ (Formula II)
wherein:
A and A′ are selected independently from one another from the group consisting of Pb, Fe, La, Y,
Gd, Bi, Ba, K, Na, K_{0.5}Bi_{0.5} and Na_{0.5}Bi_{0.5}, where, if A and A′ designate K_{0.5}Bi_{0.5} or Na_{0.5}Bi_{0.5}, then the other member of A and A′ is not chosen among K_{0.5}Bi_{0.5} and Na_{0.5}Bi_{0.5},
B and B′ are selected independently from one another from the group consisting of Ti, Sc, Al, Ga, Fe, Mn, Cr, Co, Nb,
x and y have independently from one another a value from 0 to 0.5 and the sum x+y equals a value from 0 to 0.5.

5. Method as set forth claim 1, wherein the Bismuth ferrite crystals are selected from:
BiFeO₃—PbTiO₃=(1−x)BiFeO₃+xPbTiO₃,BiFeO₃—BiScO₃=(1−x)BiFeO₃+xBiScO₃,
BiFeO₃—FeAlO₃=(1−x)BiFeO₃+xFeAlO₃, BiFeO₃—FeGaO₃=(1−x)BiFeO₃+xFeGaO₃,
BiFeO₃—FeScO₃=(1−x)BiFeO₃+xFeScO₃, BiFeO₃—LaFeO₃=(1−x)BiFeO₃+xLaFeO₃,
BiFeO₃—YFeO₃=(1−x)BiFeO₃+xYFeO₃, BiFeO₃—GdFeO₃=(1−x)BiFeO₃+xGdFeO₃,
BiFeO₃—BiMnO₃=(1−x)BiFeO₃+xBiMnO₃, BiFeO₃—BiCrO₃=(1−x)BiFeO₃+xBiCrO₃,
BiFeO₃—BaTiO₃=(1−x)BiFeO₃+xBaTiO₃, BiFeO₃—KNbO₃=(1−x)BiFeO₃+xKNbO₃,
BiFeO₃—NBT=(1−x)BiFeO₃+xNa_{1/2}Bi_{1/2}TiO₃,
BiFeO₃—KBT=(1−x)BiFeO₃+xK_{1/2}Bi_{1/2}TiO₃
where x has a numerical value from 0 to 0.4.

6. Method as set forth in claim 1, wherein the Bismuth ferrite crystals are coated with a layer containing Fe₃O₄.

7. Method as set forth in claim 1, wherein the at least one optical measurement technique is a technique where a laser beam with a first wavelength is directed onto a biological sample and a signal with a second wavelength reflected by the biological sample is detected, the second wavelength being ½ of the first wavelength and the first wavelength being in a wavelength range of preferably from 1800 to 500 nm, particularly preferably from 1640 to 1560 nm or from 1070 to 1010 nm.

8. Method as set forth in claim 1, wherein the optical measurement performed for the detection by at least one optical technique is a technique where two laser beams with a first and a second wavelength, respectively, are directed onto a biological sample and a signal with a third wavelength emitted by the biological sample is detected, the third wavelength corresponding to the sum frequency of the first two wavelengths and the first two wavelengths being in a wavelength range of preferably from 1800 to 500 nm.

9. Method as set forth in claim 1, wherein at least one optical measurement technique is a technique where a laser beam with a first wavelength is directed onto a biological sample and a signal with a second wavelength reflected by the biological sample is detected, the second wavelength being ⅓ of the first wavelength and the first wavelength being in a wavelength range of preferably from 1800 to 500 nm.

10. Method as set forth in claim 1, wherein the magnetic measurement is a technique where the biological sample is measured by MRI (“magnetic resonance imaging”) in a magnetic field with a magnetic flux density from 0.001 to 60 Tesla, preferably from 0.001 to 4 Tesla, and the biological sample is preferably imaged by the observed relaxation signals.
